(19) United States
(12) Patent Application Publication
(10) Pub. No.: US 2021/0196644 A1
(43) Pub. Date: Jul. 1, 2021

(54) CANNABINOID COATED NANO-CAPSULES

(71) Applicants: Donald Channing Cooper, Boulder, CO (US); Ana Isabel Torres-Suárez, Madrid (ES); Juan Aparicio-Blanco, Madrid (ES)

(72) Inventors: Donald Channing Cooper, Boulder, CO (US); Ana Isabel Torres-Suárez, Madrid (ES); Juan Aparicio-Blanco, Madrid (ES)

(21) Appl. No.: 17/139,846

(22) Filed: Dec. 31, 2020

Related U.S. Application Data
(60) Provisional application No. 62/955,980, filed on Dec. 31, 2019.

(51) Int. Cl. A61K 9/48; A61K 31/05; A61K 9/51; A61K 9/50

(52) U.S. Cl. CPC A61K 9/4891; A61K 31/05; A61K 9/4808; A61K 9/5192; A61K 9/5089; A61K 9/4833

(57) ABSTRACT

One of the main problems with introducing target therapeutics and diagnostic biomarkers (Target substances) into the brain is the nearly impenetrable blood brain barrier. Some of the proposed described solutions to mobilizing these target therapeutics and markers into select areas of the brain include nano-particles and nano-capsules containing at least one of the target substances with exposed cannabinoid functional end groups that can selectively facilitate nano-capsule traversal across the blood brain barrier into brain tissue. In this way, the target substances can reach their intended target within the brain to either treat or identify regions of pathology. Certain concepts described involve nano/micro-particles with exposed cannabinoid functional end groups extending beyond the surface of the nano-particles.

### CROSS-REFERENCE TO RELATED APPLICATIONS

This application claims priority to and the benefit of U.S. Provisional Patent Application No. 62/955,980 entitled: Blood Brain Barrier Penetrating Neurotheranostic Nano-capsules, filed on Dec. 31, 2019 the entire disclosure of which is hereby incorporated by reference.

### FIELD OF THE INVENTION

The present embodiments are directed to cannabinoid surface functionalized nano/micro-capsules and nano/micro-particles.

### BACKGROUND OF THE INVENTION

According to the World Health Organization, one in four people will be afflicted with psychiatric or neurological disorders at some point in their lives, placing psychiatric and neurological disorders among the leading health concerns worldwide. Though widely used, pharmaceutical treatments for psychiatric and neurological disorders are somewhat limited due to difficulties crossing into the brain via the blood-brain barrier (BBB). Likewise, dyes, fluorophores, and other pharmacological contrast agents commonly used for diagnostic evaluations are essentially out of reach for medical professionals to diagnose neurological and psychiatric disorders due to the impenetrable nature of the BBB.

FIG. 1 is a line drawing artist depiction of a human brain 100 that is well known in the prior art as viewed from the side. The human brain 100 is generally comprised of three main components; the cerebrum 109, the cerebellum 108, and the brainstem 104. The cerebellum 108 and the cerebrum 109 are used interchangeably as the “brain 100” hereinafter to simplify the description. The brain 100 is more or less composed of brain tissue 106 that is nourished by small blood vessels 102 (depicted surrounding the cerebrum 109). Brain tissue 106 is primarily composed of gray matter and white matter generally comprising neuronal, glial and endothelial cells. A skull (not shown) protects the brain 100, which is suspended in the skull by cerebrospinal fluid. The brain 100 is isolated from the bloodstream by the BBB 115, which is shown by way of example in FIG. 2 via the oval window 105 that frames a small portion of a blood vessel 102.

FIG. 2 is a line drawing of the oval window 105 magnified to graphically illustrate the approximate structures that essentially makes up the BBB 115 well known in the prior art. The BBB 115 is an interface between circulating blood 120 and the extracellular milieu of neurons and glia 116, which acts as a protective filter that essentially blocks a wide variety of systemically administered drugs from passing into the brain tissue 106 via the surrounding blood vessels 102. More specifically, the BBB 115 is a complex that surrounds most of the blood vessels 102 in the brain 100 and acts as a barrier between the bloodstream 102 and the brain's extracellular space 114. In this way, the BBB 115 essentially only permits oxygen, water and small lipid soluble substances to easily cross into the brain tissue 106 and brain's extracellular space 114. This prevents toxins and pathogens and other potentially dangerous substances from crossing into the brain 100 from the circulatory system 102. It is believed that the central components of the structure of the BBB 115 are the tight junctions 128 between the capillary endothelial cells 118, which are the cells that make up the interior surface of the blood vessels 102. Accordingly, these tight junctions 128 act as a gatekeeper restricting diffusion to selective small blood-borne substances to pass from the blood vessels 102 into surrounding brain tissue 106. Glial cells 124 also called astrocytes have astrocytic end-feet 116 that extend to and surround the blood vessel walls 112 (also known as the blood vessel basement membrane). The astrocytic end-feet 116 are also believed to form part of the BBB 115 potentially selectively allowing small blood-borne substances to pass via the small junctions 110 between the astrocytic end-feet 116.

Hence, from a therapeutic and/or diagnostic perspective, the same mechanisms that protect the brain 100 against intrusive chemicals 122 circulating in the blood vessels 105 also frustrate therapeutic interventions. Approximately, all large molecule drugs and more than 98% of small molecule drugs do not cross the BBB, which is one reason why relatively large molecular sized drugs, (e.g., growth factors and antibodies) have limited efficacy in treating diseases like Alzheimer's disease and Parkinson's disease. Accordingly, a solution to enable safe and efficient drug and/or tracer delivery across the BBB 115 in real-time is badly needed.

It is to innovations related to this subject matter that the claimed invention is generally directed.

### SUMMARY OF THE INVENTION

The present embodiments are directed to cannabinoid surface functionalized nano-capsules that efficiently pass through the BBB. The nano-capsules can contain drugs that effectively treat brain diseases and injuries and/or diagnostic markers effective in locating brain diseases and injuries. When decorated with cannabinoids, these nano-capsules efficiently pass through the BBB to deliver drugs and/or diagnostic markers directly into the brain.

With this in mind certain embodiments of the present invention are directed to nano-capsules comprising functionalized cannabinoid molecules embedded in the capsule shell. In these embodiments, the functionalized cannabinoid molecules that are exposed on the surface of the capsule provide a mechanism to efficiently migrate the nano-capsule through the BBB. These nano-capsules can also contain therapeutics (drugs, DNA, RNA, etc.) and/or diagnostic markers targeted for select brain tissue 106, such as regions of pathology for example.

In one exemplary embodiment, a capsule can comprise an in vivo dissolvable hollow shell defined by a shell thickness bound between an outer surface and an inner surface with a plurality of cannabinoid molecules are dispersed in the hollow shell. At least one of the cannabinoid molecules is at least partially disposed in the shell thickness and somewhat partially exposed at the outer surface. The capsule further includes an inner core containing excipients and an active payload, the inner core encapsulated in the hollow shell.

Another embodiment contemplates a method for moving a target substance across a blood brain barrier (BBB), the method steps include providing a nano-capsule defined by a hollow shell encapsulating an inner core containing the target substance. The hollow shell is defined by shell thickness bound between an outer surface and an inner surface. The nano-capsule is less than 1000 nm in diameter. The nano-capsule is then transported to the BBB via a blood vessel 202 (such as by way of injection, nasal, oral, etc.). When at the BBB, the nano-capsule is shuttled through the BBB into brain tissue 106 via an attractive interaction between a binding domain at the BBB and a cannabinoid functional end group that extends from the outer surface. The cannabinoid functional end group is part of a cannabinoid molecule that is at least partially embedded in the hollow shell with the functional end group sticking out from the nano-capsule surface.

Yet other embodiments of the present invention are directed to nano-capsules comprising functionalized cannabinoid molecules embedded in the capsule shell and evenly dispersed inside of the capsule core. In this way, the functionalized cannabinoid molecules that are exposed on the surface of the capsule provide a mechanism to efficiently migrate the nano-capsule through the BBB while providing both therapeutics and/or diagnostic markers targeted in addition to cannabinoids that target select brain tissue 106.

In one exemplary embodiment of cannabinoids dispersed throughout the nano-capsule, consider a capsule that generally comprises a hollow shell encapsulating an inner core wherein the hollow shell is geometrically defined by a shell thickness bound between an outer surface and an inner surface. A plurality of cannabinoid molecules is dispersed in the hollow shell and in the inner core with a fraction of the cannabinoid molecules distributed at least partially in the shell thickness and are externally exposed at the outer surface. The inner core comprising an active payload intermixed with excipients.

Another embodiment contemplates a method for manufacturing a cannabinoid capsule with functionalized cannabinoid molecules embedded in the capsule shell and evenly dispersed inside of the capsule core. The method can start with providing a base mixture of cannabinoid molecules, capsule forming molecules, excipients, and at least one active target substance. Next, the base mixture can be energized (heated and cooled, altar sonically stirred, agitated, etc.) until the capsule forming molecules generate a shell encapsulating a core. The core comprising the cannabinoid molecules, the excipients, and the at least one active target substance. Because the base mixture includes all the essential components, the cannabinoid molecules are distributed throughout the core, embedded in the shell and at least partially extend outside of a shell outer surface of the shell.

Still other embodiments of the present invention are directed to functionalized cannabinoid molecules along with therapeutics and/or diagnostic markers that are essentially evenly dispersed throughout a base substrate material forming a medicinal particle. The medicinal particle embodiment comprises functionalized cannabinoid molecules that are exposed and/or extend from the surface of the particle. The medicinal particle can be a homogenous pill, a nano-particle or something there between.

One exemplary embodiment of a cannabinoid based particle envisions a plurality of cannabinoid molecules each possessing a respective functionalized cannabinoid end group that is more or less dispersed with at least one active target substance bound together by an excipient material. The excipient material essentially binds together the cannabinoid molecules and the active target substance in an aggregate. For purposes of description, the excipient essentially defining a particle outer surface. In this configuration, a subset of the cannabinoid molecules, each positioned with the respective functionalized cannabinoid end group, extend from the particle outer surface. The particle embodiment can be a sub 2000 μm cannabinoid based particle or a nanoparticle.

While still other embodiments of the present invention are directed to conjugating a therapeutic molecule or diagnostic marker with one or more functionalized cannabinoid molecule. In this way, single therapeutic molecules or diagnostic markers can leverage the cannabinoid functional end groups to cross the BBB 115 from the circulatory system 102 into brain tissue 106.

One exemplary embodiment of the conjugated cannabinoid based molecule envisions a cannabinoid molecule chemically joined with an active target molecule. The conjugated cannabinoid based molecule has at least one functionalized cannabinoid end group at a free end of the cannabinoid molecule.

### BRIEF DESCRIPTION OF THE DRAWINGS

FIG. 1 is a line drawing artist depiction of a human brain that is well known in the prior art as viewed from the side;

FIG. 2 is a line drawing of the oval window from FIG. 1 magnified to artistically illustrate the approximate structures that essentially makes up the BBB that is also well known in the prior art;

FIG. 3A is a line drawing of cross-sectioned cannabinoid decorated nano-capsule consistent with embodiments of the present invention;

FIG. 3B illustratively depicts a cross-section view of an optional cannabinoid decorated nano-capsule embodiment consistent with embodiments of the present invention;

FIG. 4A is a line drawing cross-section view of a cannabinoid embedded nano-capsule shell consistent with embodiments of the present invention;

FIG. 4B is a line drawing of yet another nano-capsule embodiment illustratively depicting a nano-capsule cross-section with organized cannabinoid molecules dispersed in the outer shell with functional cannabinoid and groups exposed consistent with embodiment of the present invention;

FIG. 4C illustratively depicts a cross-section line drawing of cannabinoid molecules dispersed throughout the capsule shell and the nano-particles core consistent with embodiments of the present invention;

FIG. 5 is a line drawing of a cannabinoid enriched nano-particle consistent with embodiments of the present invention;

FIGS. 6A-6N are line drawing diagrams showing the conventional organic chemical structure of 14 common cannabinoids that can be advantageously used in embodiments of the present invention; and

FIGS. 7A-7D are line drawing diagrammatic illustrations of at least one cannabinoid molecule joined or otherwise conjugated with a target molecule consistent with embodiments of the present invention.

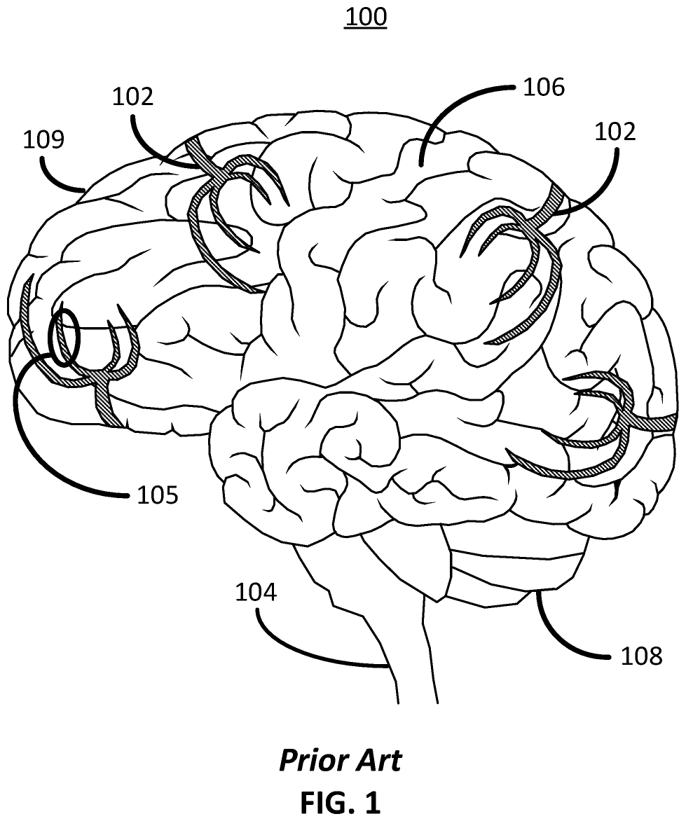

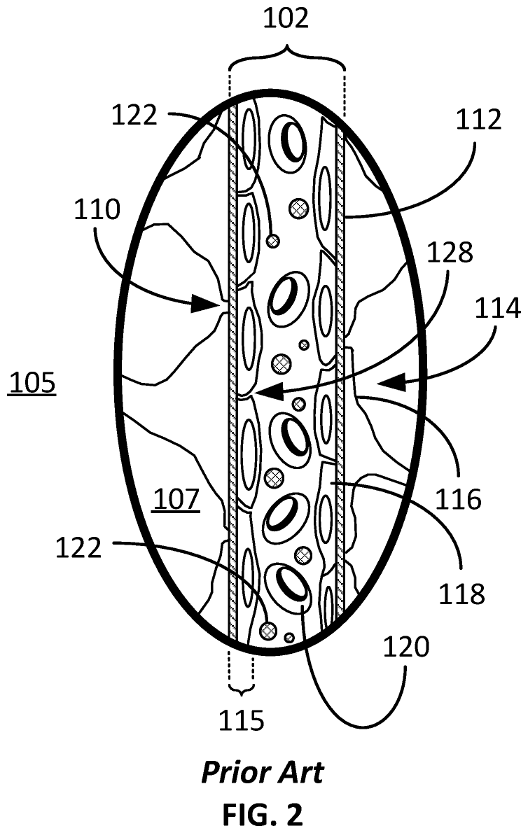

### DETAILED DESCRIPTION

Initially, this disclosure is by way of example only, not by limitation. Thus, although the instrumentalities described herein are for the convenience of explanation, shown and described with respect to exemplary embodiments, it will be appreciated that the principles herein may be applied equally in other types of situations involving similar uses of nano and micro-particles. The phrases “in one embodiment”, “according to one embodiment”, and the like generally mean the particular feature, structure, or characteristic following the phrase is included in at least one embodiment of the present invention, and may be included in more than one embodiment of the present invention. Importantly, such phases do not necessarily refer to the same embodiment. If the specification states a component or feature “may”, “can”, “could”, or “might” be included or have a characteristic, that particular component or feature is not required to be included or have the characteristic. In what follows, similar or identical structures may be identified using identical callouts.

One of the main problems with introducing target therapeutics and markers (target substances) into the brain is the nearly impenetrable BBB. Some of the proposed described solutions to mobilizing these target therapeutics and markers into select areas of the brain include nano-particles and nano-capsules containing at least one of the target substances with exposed cannabinoid functional end groups that can facilitate migration across the BBB into brain tissue. In this way, the target substances can reach their intended target within the brain to either treat or identify regions of pathology. Certain concepts described herein involve nano/micro-particles with exposed cannabinoid functional end groups extending beyond the surface of the nano-particles.

Certain embodiments of the present invention are directed to nano-capsules decorated or otherwise coated with functionalized cannabinoid molecules that efficiently pass through the BBB 115 directly into the brain 100. The nano-capsules can contain therapeutics (drugs, DNA, RNA, etc.) to treat brain diseases and injuries and/or diagnostic markers to pinpoint/locate brain diseases and injuries.

Other embodiments of the present invention are directed to nano-capsules comprising functionalized cannabinoid molecules embedded in the capsule shell. In these embodiments, the functionalized cannabinoid molecules that are exposed on the surface of the capsule provide a mechanism to efficiently migrate the nano-capsule through the BBB. These nano-capsules can also contain therapeutics (drugs, DNA, RNA, etc.) and/or diagnostic markers targeted for select brain tissue 106, such as regions of pathology for example.

Still other embodiments of the present invention are directed to functionalized cannabinoid molecules along with therapeutics and/or diagnostic markers that are essentially evenly dispersed throughout a base substrate material forming a medicinal particle. The medicinal particle embodiment comprises functionalized cannabinoid molecules that are exposed and/or extend from the surface of the particle. The medicinal particle can be a homogenous pill, a nano-particle or something there between.

Yet other embodiments of the present invention are directed to conjugating a therapeutic molecule or diagnostic marker with one or more functionalized cannabinoid molecule. In this way, single therapeutic molecules or diagnostic markers can leverage the cannabinoid functional end groups to cross the BBB 115 from the circulatory system 102 into brain tissue 106.

Though nano-capsules and nano-particles are used in the present description as a vehicle for an active ingredient payload 208, micro-capsules can be equally used as a delivery system for organs that do not require nano-particles without departing from the scope and spirit of the present invention. Nano-capsules are defined as smaller than 1000 nm, but are typically under 100-500 nm. Micro-capsules are generally considered to be in a range between 1 to 2000 μm.

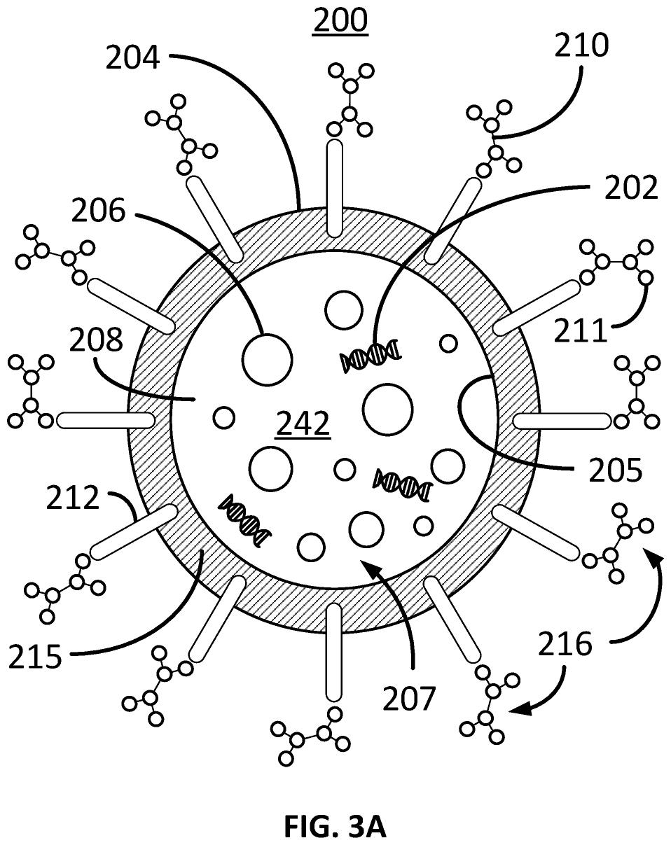

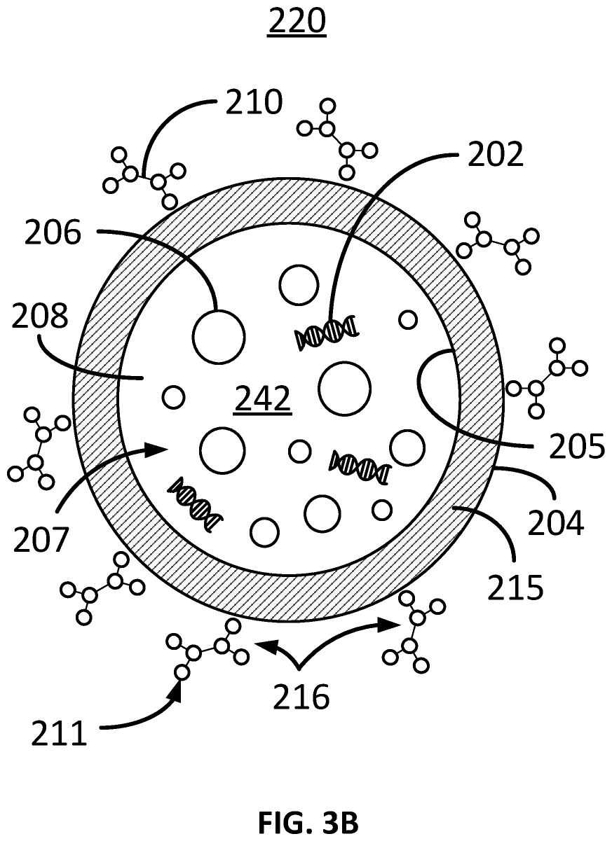

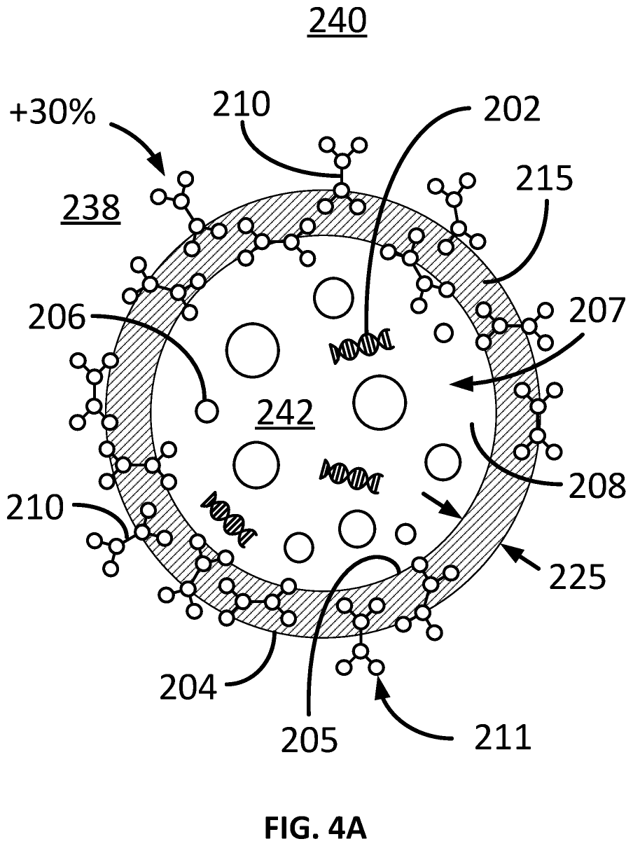

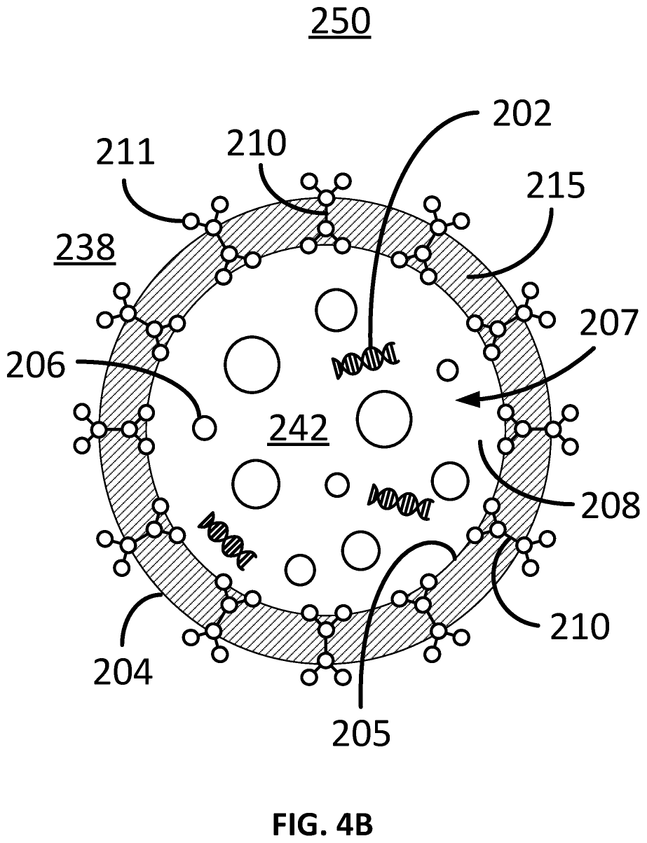

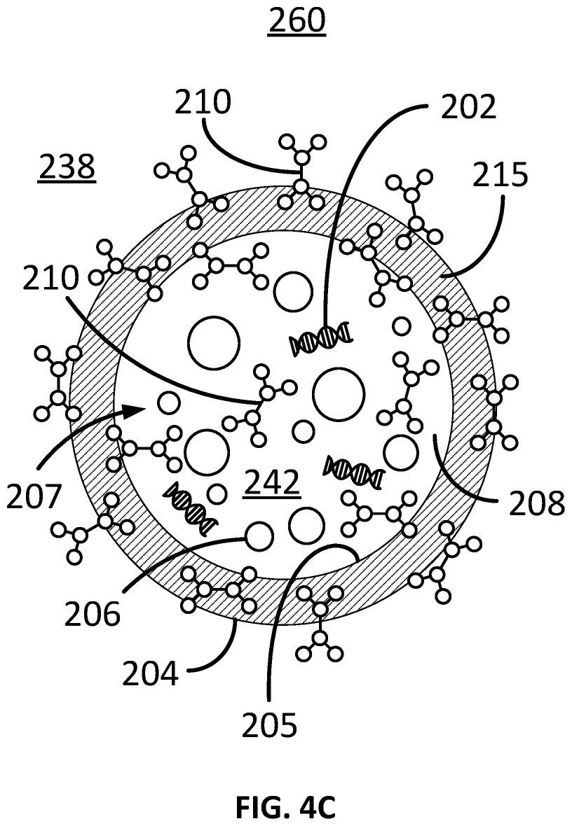

FIG. 3A is a line drawing of cross-sectioned cannabinoid decorated nano-capsule consistent with embodiments of the present invention. The decorated nano-capsule 200 generally comprises an environmental-specific dissolvable outer hollow shell 215, a target substance 208 inside of the hollow shell 215, and a cannabinoid coating 216 decorating the outside of the hollow shell 215. The term “environmental-specific dissolvable” as used herein, means dissolvable in a specific targeted environment, such as a specific environment within a body, specific tissue within the body, a specific organ within the body, etc. The environmental-specific dissolvable hollow shell 215 is a shell our boundary defined between an outer surface 204 and an inner surface 205. The dissolvable outer shell 215 can be a biodegradable polymer membrane, such as a biodegradable polyester or surfactant, useable in biological systems. Examples of the dissolvable outer shell 215 includes a family of synthetic polymers, such as Poly-e-caprolactone (PCL), poly (lactide) (PLA), poly (lactide-co-glicolide) (PLGA), Thiolated poly (methacrylic acid) and poly (N-vinyl Pyrrolidone), as well as a host of naturally occurring polymers, such as chitosan, gelatin, sodium alginate, and albumin. Chitosan, gum arabic, pectin, carrageenan, alginates, and carboxymethyl cellulose (CMC) are embodiment examples of natural materials for the formation of food-grade nano-capsule shells. Other surfactants include polysorbates, which are the ethoxylated sorbitan esters and are manufactured by the reaction of sorbitol with various fatty acids. Three examples of polysorbate are polysorbate 20, 60, and 80, which utilize lauric, stearate, and oleate, respectively, for the fatty acid portion of the molecule, which forms the shell interface between the lipid core and the aqueous environment 238. Other nano-capsule shells 215 can include liposomes, along with polysaccharides and saccharides. The dissolvable outer shell 215 as described in conjunction with FIG. 3A can be applied to the other nano-capsule embodiments disclosed in conjunction with FIGS. 4A-4C (minus the organization and location of the cannabinoid molecules 210) below without departing from the scope and spirit of the present invention.

Lipid nano-capsules provide an exemplary environment in which certain embodiments of the present invention involving a capsule shell can be practiced. However, as discussed above, a skilled artisan will appreciate that concepts disclosed herein can equally be used with a host of naturally occurring or synthetic nano-capsules or particles. In other words, the concepts disclosed are not limited to lipid capsules or lipid particles (that are not capsules). Accordingly, certain embodiments contemplate a hollow surfactant shell 215 (a lipid crystallization of nanoemulsion template) formed or otherwise made out of nanoemulsion droplets that adopt (i.e., seek the lowest energy point) the form of nano-capsules. In certain embodiments, the hollow shell 215 is rigid. Surfactants are well suited for nano-capsules because surfactant molecules spontaneously bond (organize) with one another to form sealed bubbles. Surfactants exhibit well-known properties of lowering the surface tension between disparate phase materials, such as between oil and water, for example. Surfactants are amphiphilic structures often defined by molecule arrangements with hydrophilic heads and hydrophobic tails (that are inside of the surfactant ball). There are a variety of techniques to form nano-capsules from surfactants including phase inversion composition methods, phase inversion temperature methods, high pressure homogenization methods or sonication just to name a few. Accordingly, surfactant bubbles that make up the hollow surfactant shell 215 combined with a lipid core 242 provide an excellent environment to practice aspects of the present invention.

One exemplary technique for creating a lipid nano-capsule is by way of the inversion temperature method. Formulating a lipid nano-capsule using this technique can start with mixing together all excipient ingredients 207, which can include aqueous and oily substances along with surfactants and other non-active ingredients. Excipients are inactive substances that serve as an oily or surfactant vehicle or medium for a drug or other pharmacologically active substance. Excipients are things like coloring agents, preservatives, and fillers. Excipients can include any number of non-active pharmaceutical or target ingredient, such as terpenes, di-terpenes, etc. Though the cannabinoid molecules 210 are distinguished herein to draw particular attention thereto, the scientific perspective on the use of cannabinoid molecules 210 as used herein is that they can be viewed as an excipient that enhance bioavailability of the active/target pharmacological substances. The mixed excipients 207 plus the active ingredients 208 are progressively heated over the phase inversion temperature and then gradually cooled down below the phase inversion temperature thereby forming the capsules. The heating and cooling step can be repeated over at least one more cycle. Lastly, the mixture is reheated over the phase inversion temperature and then quenched in cold water to obtain a suspension of the lipid nano-capsules. Hence, at this point the nano-capsule comprises the target substance 208 inside of the hollow shell 215.

Another exemplary technique for creating lipid nano-capsules is to simply homogenize the excipients 207 and active/target substances 208. When homogenized, the surfactants naturally self-organize into nano-capsules or nano-bubbles 215 trapping the active ingredients 208 and other excipients 207 therein.

Another exemplary technique for creating a lipid nano-capsule is by way of using high-energy sonication. The process involving growth and collapse of micro bubbles (cavitation) in a liquid medium by way of high-intensity ultrasound using energy ranging 0.5-2 W/mL power density for various durations, such as between 10 s and 30 minutes (1-5 s ON and 5-10 s OFF mode), can be accomplished at temperatures between 0 C.° and 80 C.° using an ice bath or water circulation. Ultrasonic frequencies of between 20 kHz-40 kHz can be employed for producing micro-jets and turbulence in the liquid. These intense shear forces induce disruption at the interface of immiscible phases, thereby, facilitating the production of fine and stable emulsions in the presence of surfactants. Formulating a lipid nano-capsule using this technique can start with mixing together all excipient ingredients 207, which can include aqueous and oily substances along with surfactants and other non-active ingredients.

The target substance 208, also called the payload, can be a liquid, either oil-based or water-based, or optionally a solid or gas. The payload can include one or more therapeutic medicines, genetic material (such as antibodies, pieces of genes, new genetic code, etc.), dyes, radiographic markers or some combination thereof.

With specific reference to FIGS. 3A and 3B, after forming the nano-particles (i.e., capsule 215 and payload 208), a cannabinoid coating is applied to the nano-particle surface 204. Certain embodiments envision bathing the nano-particles in a liquid cannabinoid mixture that contains one or more different cannabinoid molecules 210 suspended in either an oil-based or aqueous-based liquid. Another embodiment of a cannabinoid-coating application method includes sonicating the pre-formed non-coated nano-particles in a surfactant shell forming solution containing cannabinoids at concentrations that may range between 2 mg/mL and 100 mg/mL.

Certain embodiments contemplate an intermediate molecule 212 that conjugates or otherwise links the cannabinoid molecule 210 to the capsule surface 204. For example, if the capsule surface 204 is primarily comprised of hydrophilic end groups and the cannabinoid 210 is hydrophobic, then the intermediate molecule 212 may have a hydrophilic end attracted to the capsule surface 204 and a hydrophobic end attracted to the cannabinoid 210. Of course, other techniques, such as cannabinoid vapor deposition or other similar techniques can be used to decorate the capsule surface 204 without departing from the scope and spirit of the present invention. Regardless of the technique applying the cannabinoid molecule 210 to the nano-capsule 215, functionalized cannabinoid end groups 211 are exposed to cooperate or otherwise interact with cellular structures and molecular signaling cascades in the human body, such as cannabinoid (CB1 and 2), serotonergic and transient receptor potential vanilloid transmembrane receptors in the human brain 100.

FIG. 3B illustratively depicts a cross-section view of an optional cannabinoid decorated nano-capsule embodiment consistent with embodiments of the present invention. The cannabinoid decorated nano-capsule 220 does not comprise or leverage the use of an intermediate binding molecule 212, rather the cannabinoid molecules 210 are dispersed whether evenly or otherwise on the nano-particles surface 204. Whether by way of an intermediate molecule 212 or not, the end result is a lipid nano-capsule with a functionalized cannabinoid coating 216 (i.e., with functionalized cannabinoid end groups 211 extending from the surface 204), as shown. The cannabinoid decorated nano-capsule 220 can be made by subjecting the nano-capsule 215 to a cannabinoid solution, cannabinoid vapor, or other cannabinoid environment understood by those skilled in the art.

The exemplary nano-capsules embodiments 200 and 220 (as well as the other embodiments disclosed below in conjunction with FIGS. 4A-4C, 5 and 7A-7D) can be delivered to the BBB 115 in a living animal or human by way of injecting an aqueous solution of nano-capsules 200 or 220 in a blood vessel 102. Optional embodiments envision anhydrous self-emulsifying fatty acid containing cannabinoid-coated nano-capsules that are formed immediately upon contact and dispersion in the aqueous acidic stomach fluid and are absorbed from the villi of the small intestine into the circulating blood 200 or 220 into a lymphatic vessel (not shown).

Unlike blood vessels 102, lymphatic vessels do not pass through the liver thereby avoiding the process of filtering out and metabolizing at least some of the nano-capsules 200 or 220. Accordingly, certain embodiments contemplate moving the nano-capsule embodiments 200 or 220 into brain tissue 106 via the lymphatic system. In vivo introduction of the nano-capsule embodiments 200 and 220 into brain tissue 106 via the BBB 115 can be accomplished by way of injecting a solution of nano-capsules 200 and 220 into a blood vessel 102 or via oral or intranasal or transdermal delivery. Oral delivery facilitates the nano-capsules 200 and 220 reaching the bloodstream 102 via the stomach or the intestinal tract. Other embodiments envision intranasal application of 0.1-1 mL volume of cannabinoid-coated nano-capsules via spray-pump, nebulized or metered-dose aerosolized inhaler. In this case nano-capsules are absorbed across the nasal epithelium into the brain vasculature without entering the general circulation or passing through the liver to be inactivated. Other embodiments envision transdermal application in the form of a cream or patch applied to the skin whereby the nano-capsules are capable of permeating the skin for absorption into the blood stream.

Optionally, the exemplary nano-capsules 200 or 220 can be manually introduced to endothelial cells 118 (of a living creature) that form part of a BBB 115 via a blood vessel 102. Certain embodiments envision the nano-capsules 200 or 220 cooperating with the transmembrane protein that has a binding domain for the cannabinoid (such as CBD) that shuttles the nano-capsules 200 across the barrier. There may be binding elements on either side or on both the blood facing and brain facing sides of the endothelial cells 118. Accordingly, transportation of the nano-capsule 200 and 220 across the BBB 115 is facilitated due to the cannabinoid active end groups exposed on the nano-capsule surface 204 coupled with the extremely small form factor (sub 1000 nm size) of the nano-capsule. More plainly, certain embodiments contemplate that the cannabinoid molecules 210 ‘enhance’ passageway from the blood vessel 102 into the brain tissue 106 via the BBB 115 and yet in other embodiments the cannabinoid molecules 210 ‘facilitate’ passageway from the blood vessel 102 into the brain tissue 106 via the BBB 115.

The cannabinoid coating 216 decorating the outside of the hollow shell 215 (containing the payload 208 in the core 242) can be applied in a number of ways so long as the end result is a lipid nano-capsule with a functionalized cannabinoid coating 216, as shown. Importantly, the cannabinoid coating 216 is not embedded in the structure of the hollow shell 215, rather it is simply a coating 216 on top of the outer surface 204 of the hollow shell 215. Some embodiments envision the cannabinoid coating 216 being essentially a monolayer of cannabinoid molecules 210 while other embodiments envision the cannabinoid coating 216 being a multilayer of cannabinoid molecules 210. Still other embodiments envision a cannabinoid molecule 210 decorated outer shell surface 204 with an insufficient quantity of cannabinoid molecules 210 to completely coat the outer surface 204. The term decorated is defined as either a single molecular layer of cannabinoid molecules 210 or a sparse coating of cannabinoid molecules 210 that do not fully coat the nano-capsule 200 or 220. In other words, the term decorated is (at the most) a single molecular layer or less than a single layer of cannabinoid molecules 210.

FIG. 4A is a line drawing cross-section view of a cannabinoid embedded nano-capsule shell consistent with embodiments of the present invention. As shown, the nano-capsule 240 comprises a payload 208 in the core 242 surrounded by an outer shell 215 comprising embedded cannabinoid molecules 210. The interior payload 208 is essentially devoid of a complete cannabinoid molecule 210. In the case of a surfactant outer shell 215, which in certain embodiments is one surfactant molecule thick, the embedded cannabinoid molecules 210 may be partially in the nano-particles core 242 poking through the inner cannabinoid surface 205, as shown. In other embodiments, the embedded cannabinoid molecules 210 are exposed or otherwise poke through the outer capsule surface 204 into the outside environment 238. Certain embodiments envision that some of the cannabinoid molecules 210 extend from the outer shell 215 ‘poking out’ as much as 30% with the remainder (70%) embedded in the outer shell 215. In some embodiments, the cannabinoid molecules 210 extend from the outer shell 215 ‘poking out’ as much as 95% with the remainder embedded in the outer shell 215, as shown. In this way, functionally active cannabinoid molecule end groups 211, which are exposed can play an active role in passing through the BBB 115 and/or actively contribute to cooperating with cannabinoid receptors in the human body or inside an animal. With regards to the capsule core 242, the payload 208 can be mixed or otherwise supported by any number of different materials, such as a lipid core, for example.

FIG. 4A illustratively depicts the cannabinoid molecules 210 randomly dispersed in the outer shell 215, however as shown in FIG. 4B another nano-capsule embodiment 250 illustratively depicts a line drawing of a nano-capsule cross-section with organized cannabinoid molecules 210 dispersed in the outer shell 215 with functional cannabinoid and groups 211 exposed. For example, a surfactant outer shell may be a highly organized bubble that is essentially a single surfactant molecule deep with cannabinoid molecules 210 tightly packed and arranged between at least some of the surfactant molecules, as shown. The cannabinoid functional end groups 211 of the highly organized (or in some cases, a somewhat organized or less than highly organized) cannabinoid molecules 210 are extending from, or ‘poking out’, of the outer shell surface 204 in the outside environment 238, as shown. In this way, the cannabinoid functional and groups 211 can cooperate with cannabinoid receptors at or on the other side of the BBB 115, for example.

Accordingly, certain embodiments envision the fabrication of BBB traversable nano-capsules by mixing cannabinoid containing or non cannabinoid containing lipid soluble solutions with surfactants using temperature phase-inversion methods. Other embodiments envision the fabrication of BBB traversable cannabinoid surface decorated or capsule shell integrated nano-capsules by mixing lipid soluble solutions with cannabinoid-containing surfactants using temperature phase-inversion methods.

With the present description of FIGS. 4A and 4B in mind, below are some examples of certain embodiments illustratively complementing some of the methods and apparatus concepts to aid the reader. The elements called out below are examples provided to assist in the understanding of the present invention and should not be considered limiting.

In that light, one embodiment of the present invention envisions a capsule 240 or 250 comprising: an in vivo dissolvable hollow shell 215 defined by a shell thickness 225 (which can be a monolayer of surfactant molecules, for example) bound between an outer surface 204 and an inner surface 205; a plurality of cannabinoid molecules 210 dispersed in the hollow shell 215, at least one of the cannabinoid molecules 210 is at least partially disposed in the shell thickness 225 and at least partially exposed at the outer surface 204; an inner core 242 containing excipients 207 and an active payload 208, the inner core 242 encapsulated in the hollow shell 215.

The capsule embodiment 240 or 250 further imagines the at least one cannabinoid molecule 210 comprising a functionalized cannabinoid end group 211 that is at least partially exposed at the outer surface 204.

The capsule embodiment 240 or 250 further can comprise at least a second of the cannabinoid molecules 210 comprising a functionalized cannabinoid end group 211 extending from the outer surface 204.

Optionally, the capsule embodiment 240 or 250 further can comprise at least a second of the cannabinoid molecules 210 extending inwardly from the inner surface 205.

The capsule embodiment 240 or 250 further ponders wherein the hollow shell 215 is essentially composed of a surfactant mixed with the cannabinoid molecules 210.

The capsule embodiment 240 or 250 further considers wherein the active payload 208 is selected from a group comprising at least one of a pharmaceutical drug, a marker, and a genetic nucleic acid molecular sequence 202 (such as DNA, RNA, etc.). This embodiment can be expanded wherein the marker is selected from a group consisting of a radioactive tracer, a dye, and a fluorescent dye.

The capsule embodiment 240 or 250 further imagines wherein the capsule 240 is a nano-capsule that has a diameter (or optionally the largest measurement across the capsule) that is less than 1000 nm in diameter, while other embodiments imagine the capsule 240 or 250 having a diameter (or largest trans measurement across the capsule) being a micro-capsule that is between 1 μm and 2000 μm.

The capsule embodiment 240 or 250 further envisions wherein the excipients 207 is primarily comprised of lipids.

The capsule embodiment 240 or 250 further contemplates wherein the plurality of cannabinoid molecules 210 are geometrically organized within the hollow shell 215 wherein at least some of the cannabinoid molecules 210 extend from the outer surface 205 exposing functionalized end groups 211 of the cannabinoid molecules 210. Geometrically organized cannabinoid molecules 210 are envisioned organizing spatially within highly organized capsule shell molecules, such as surfactant molecules that in certain embodiments make up the capsule shell 215. Accordingly, the cannabinoid molecules 210 themselves by de facto would have to organize geometrically in order to fit in between the highly organized molecules that make up the capsule shell 215.

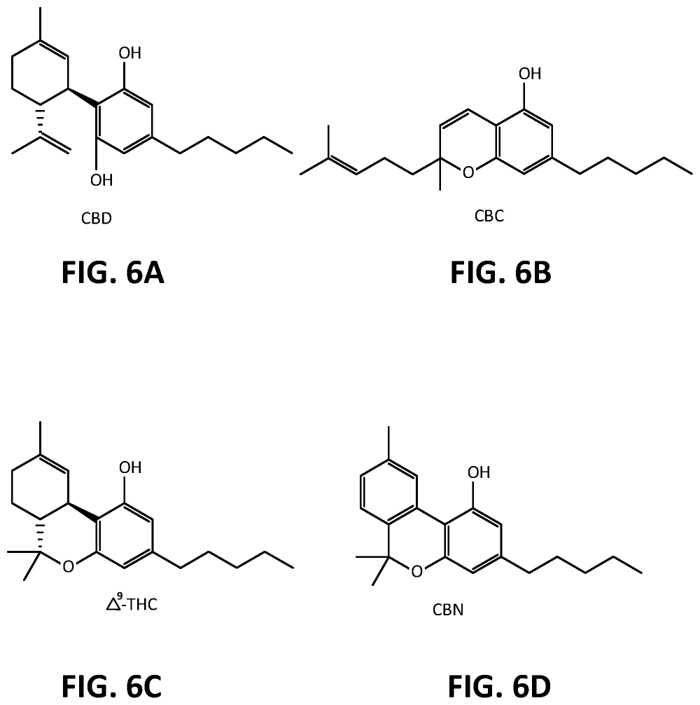

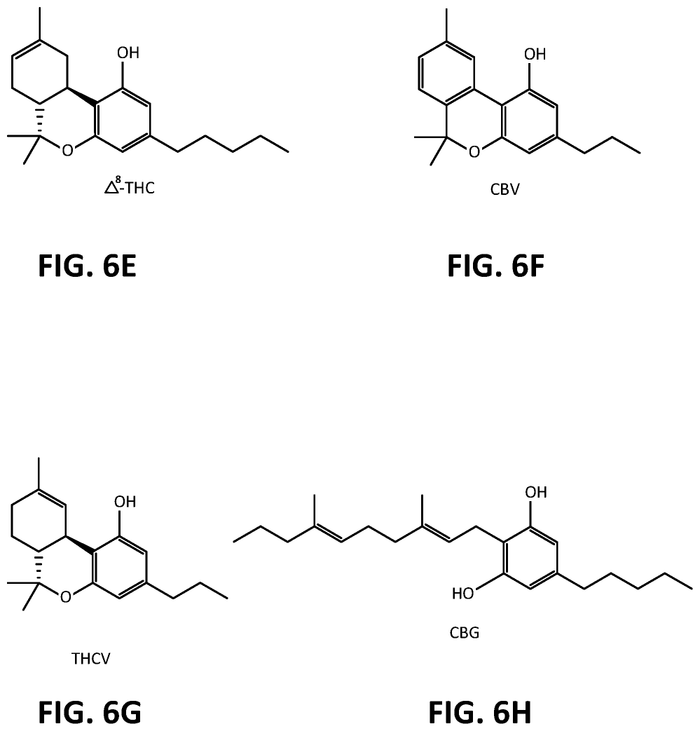

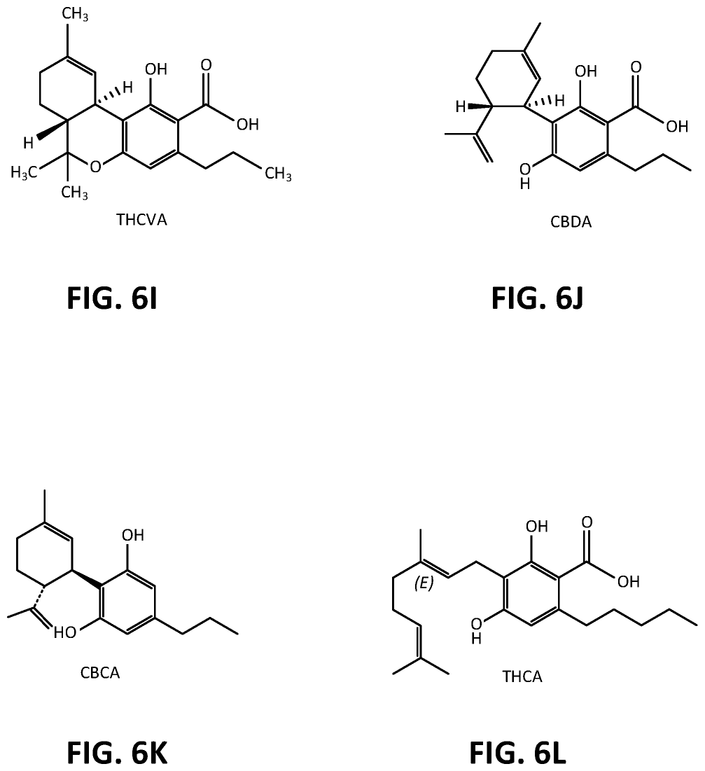

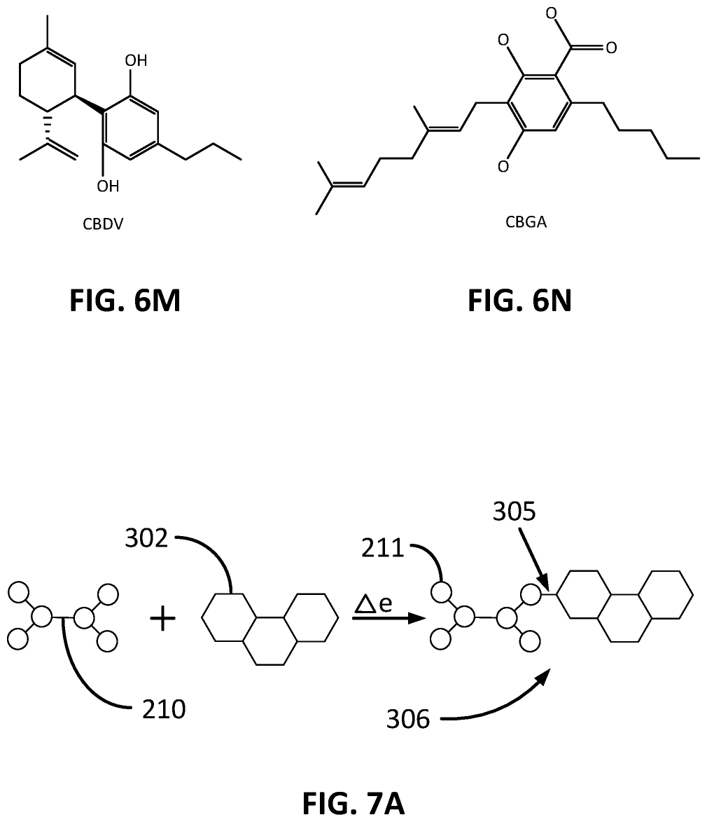

The capsule embodiment 240 or 250 further considers wherein the cannabinoid molecules 210 are CBD, shown in FIG. 6A.

In certain capsule embodiments 240 or 250, none of the cannabinoid molecules 210 are entirely in the inner core 242. The cannabinoid molecules 210 can be partially in the inner core 242 by virtue of poking through the inner shell surface 205, but not entirely in the inner core 224.

Certain other embodiments contemplate a method for moving a target substance 208 across a BBB 115, the method comprising: providing a nano-capsule 240 or 250 defined by a hollow shell 215 encapsulating an inner core 242 containing the target substance 208, the hollow shell 215 defined by shell thickness 225 bound between an outer surface 204 and an inner surface 205, the nano-capsule less than 1000 nm in diameter. The nano-capsule 240 or 250 is then transported to the BBB 115 via a blood vessel 202, e.g., by injection into a blood vessel or lymphatic vessel, absorption through a nasal/sinus membrane, via epidermal exposure, orally taken and absorbed in the G.I. tract, etc. Once at the BBB 115, shuttling the nano-capsule 240/250 through the BBB 115 into brain tissue 106 via an attractive interaction between a binding domain at the BBB 115 and a cannabinoid functional end group 211 that extends from the outer surface 204, the cannabinoid functional end group 211 is part of a cannabinoid molecule 210 that is at least partially embedded in the hollow shell 215. Certain embodiments contemplate that at least 30% of the cannabinoid molecule 210 is embedded in the shell thickness 225.

The method embodiment can further comprise dissolving the hollow shell 215 in the brain tissue 106 after the shuttling step.

Optionally the method embodiment further contemplates wherein the hollow shell 205 is a surfactant bubble. A surfactant bubble is based on the low energy result of the surfactant molecules aligning and connecting.

The method embodiment can further be wherein the target substance 208 is selected from a group comprising at least one of a pharmaceutical drug, a marker, and a genetic nucleic acid molecular sequence 202. This can further be wherein the marker is selected from a group consisting of a radioactive tracer, a dye, and a fluorescent dye.

In another embodiment, the nano-capsule 240 or 250 can comprise a hollow shell 215 containing an inner core 242; a plurality of cannabinoid molecules 210 each comprising a functionalized cannabinoid end group 211, at least one of the cannabinoid molecules 210 extending through the hollow shell 215 with the functionalized cannabinoid end group 211 exposed outside of a shell outer surface 204 of the hollow shell 215; the inner core 242 comprising an active payload 208 intermixed with excipients 207.

The nano-capsule embodiment 240 or 250 further considers wherein the cannabinoid molecules 210 comprise more than one functional end group.

The nano-capsule embodiment 240 or 250 further contemplates wherein the hollow shell 215 is defined by a shell thickness 225 bound between an outer surface 204 and an inner surface 205 and wherein at least 20% of the cannabinoid molecules 210 (embedded in the hollow shell 215) are embedded at least 30% in the shell thickness 215 with the corresponding functionalized cannabinoid end group 211 extending outwardly from the shell outer surface 204 the remaining 70% of the cannabinoid molecules 210.

The above sample embodiments loosely directed to FIGS. 4A and 4B should not be considered limiting to the scope of the invention whatsoever because many more embodiments and variations of embodiments are easily conceived within the teachings, scope and spirit of the instant specification.

FIG. 4C illustratively depicts a cross-section line drawing of cannabinoid molecules dispersed throughout both the capsule shell and the nano-particles core. As shown, the cannabinoid molecules 210 are dispersed throughout (an in some cases, somewhat evenly) the nano-particle core 242 and capsule shell 215 wherein the cannabinoid molecules 210 are exposed and even extending from the nano-capsule shell outer surface 204 into an external environment 238. In this configuration, the cannabinoid functional and groups 211 are arranged to cooperate with cannabinoid receptors at or on the other side of the BBB 115, for example. A skilled artisan will appreciate that the cannabinoid molecules 210 integrated with the nano-capsule may be organized similarly in the capsule shell 215 of FIG. 4B. In this configuration, the cannabinoid molecules 210, the surfactant molecules and the other excipient ingredients 207 as well as the active/target ingredients can all be mixed together and then processed, such as by ultrasonically homogenizing the combination into nano-particles 240, 250 and 260, as shown. This may offer manufacturing benefits to the previous embodiments discussed.

With this in mind, certain embodiments of the present invention therefore contemplate some excipients comprising an emulsion formed upon mixing linoleic mono-di and triglycerides that may or may not contain polyethylene glycol esters of fatty acids together with a surfactant in an aqueous vehicle to form lipid-core micro-capsules and/or nano-capsules with a surfactant shell. Optionally, various combinations of lipid-soluble phytochemicals, such as cannabinoids, sesquiterpenes, monoterpenes, diterpenes at concentrations up to 90% by weight may be added to the glyceride and fatty acid containing lipid-core. Similarly, a surfactant composed of hydrophilic and oleophilic groups may be mixed with surfactant soluble phytochemicals like cannabinoids, sesquiterpenes, monoterpenes, diterpenes at concentrations up to 90% by weight and added to the lipid-core solution and mixed in an aqueous vehicle forms a shell surrounding a nano-capsule that expose cannabinoid chemical moieties of the added phytochemicals 210 on the surface of the nano-capsule that interact with and cross the brain vasculature 102 comprising the BBB 115.

Below are some embodiment examples to illustratively complement certain aspects of the invention as it relates to FIG. 4C. The elements called out below are examples provided to assist in the understanding of the present invention and should not be considered limiting.

In that light, one embodiment of the present invention envisions a capsule 260 comprising: a hollow shell 215 encapsulating an inner core 242, the hollow shell 215 defined by a shell thickness 225 bound between an outer surface 204 and an inner surface 205; a plurality of cannabinoid molecules 210 dispersed in the hollow shell 215 and in the inner core 242, a fraction of the cannabinoid molecules 210 are distributed at least partially in the shell thickness 225 and are externally exposed at the outer surface 204; and the inner core 242 comprising an active payload 208 intermixed with excipients 207. Externally exposed means that a portion of the cannabinoid molecules 210 stick out from the capsule outer surface 204 and are exposed to the environment 238 outside of the capsule outer surface 204.

The capsule embodiment 260 further imagines at least one of the cannabinoid molecules 210 comprising a functionalized cannabinoid end group 211 extending from the outer surface 204.

The capsule embodiment 260 further can comprise at least a second of the cannabinoid molecules 210 comprising a functionalized cannabinoid end group 211 extending from the outer surface 204.

Optionally, the capsule embodiment 260 further contemplates wherein the hollow shell 215 is essentially composed of a surfactant mixed with the cannabinoid molecules 210.

The capsule embodiment 260 further ponders wherein the active payload 208 is selected from a group comprising at least one of a pharmaceutical drug, a marker, and a genetic nucleic acid molecular sequence 202.

The capsule embodiment 260 further considers wherein the active payload 208 is selected from a group comprising at least one of a pharmaceutical drug, a marker, and a genetic nucleic acid molecular sequence 202 (such as DNA, RNA, etc.). This embodiment can be expanded wherein the marker is selected from a group consisting of a radioactive tracer, a dye, a fluorescent dye.

The capsule embodiment 260 further imagines wherein the capsule 240 is a nano-capsule that has a diameter (or optionally the largest measurement across the capsule) that is less than 1000 nm in diameter, while other embodiments imagines the capsule 240 or 250 that has a diameter (or largest trans measurement across the capsule) being a micro-capsule that is between 1 μm and 2000 μm.

The capsule embodiment 260 further envisions wherein the excipients 207 is primarily comprised of lipids.

The capsule embodiment 260 further contemplates wherein the plurality of cannabinoid molecules 210 are geometrically organized within the hollow shell 215 wherein at least some of the cannabinoid molecules 210 extend from the outer surface 205 exposing functionalized end groups 211 of the cannabinoid molecules 210.

The capsule embodiment 260 further considers wherein the cannabinoid molecules 210 are CBD, as shown in FIG. 6A, or optionally the cannabinoid molecules 210 are made up of at least two different types of cannabinoid molecules, such as CBD and CBC of FIG. 6B.

In certain capsule embodiments 260, a plurality of the cannabinoid molecules 210 comprise a first cannabinoid molecule portion embedded in the capsule 240 and a second cannabinoid molecule portion extending from the outer surface 204, the second cannabinoid molecule portion includes a functionalized cannabinoid end group 211.

Certain other embodiments contemplate a method for manufacturing a cannabinoid capsule 260. The method can comprise providing a base mixture of cannabinoid molecules 210, capsule forming molecules, excipients 207, and at least one active target substance 208. Next, energizing the base mixture until the capsule forming molecules generate a shell 215 encapsulating a core 242, the core 242 comprising the cannabinoid molecules 210, the excipients 207, and the at least one active target substance 208. The end result is that the cannabinoid molecules 210 are distributed throughout the core 242, embedded in the shell 215, and at least partially extend outside of a shell outer surface 204 of the shell 215.

The method embodiment can further comprise performing the energizing step until the shell 215 is less than 1000 nm in diameter.

Optionally the method embodiment further contemplates the cannabinoid molecules 210 each comprise a functionalized cannabinoid end group 211, at least some of the functionalized cannabinoid end groups 211 extend outside of the shell outer surface 204.

The method embodiment can further be wherein the cannabinoid molecules 210 comprise at least two different types of cannabinoids (see FIGS. 6A-6N).

The method embodiment can further include wherein the capsule forming molecules are selected from a group consisting of Poly-e-caprolactone (PCL), poly (lactide) (PLA), poly (lactide-co-glicolide) (PLGA), Thiolated poly (methacrylic acid), poly (N-vinyl Pyrrolidone), chitosan, gelatin, sodium alginate, and albumin, gum arabic, pectin, carrageenan, alginates, and carboxymethyl cellulose (CMC), liposomes, surfactants, polysaccharides, and saccharides.

The method embodiment can further contemplates wherein the energizing step includes sonicating the base mixture, heating and cooling the base mixture over multiple cycles, agitating the base mixture, and subjecting the base mixture to acid (such as stomach acid in vivo or in a container in vitro). Accordingly, one embodiment of the present invention envisions BBB traversable cannabinoid surface activated self-emulsifying lipid and surfactant mixtures that are capable of spontaneous formation of blood circulation absorbable micro-capsules and nano-capsules upon contact and dispersion into acidic (less than pH 3) hydrochloric acid containing gastric juices.

Still, yet other embodiments a cannabinoid based capsule 260 comprising an inner core 242 encapsulated by a biodegradable hollow shell 215 and a plurality of cannabinoid molecules 210 dispersed in both the hollow shell 215 and in the inner core 242. Each of the cannabinoid molecules 210 comprise a functionalized cannabinoid end group 211 wherein a portion of the cannabinoid molecules 210 are distributed at least partially in hollow shell 215. Some of the portion of the cannabinoid molecules 210 extend from an outer shell surface 204 of the hollow shell 215. The inner core 242 comprising an active payload 208 intermixed with excipients 207.

The cannabinoid based capsule embodiment 260 further considers the hollow shell 215 is defined by a shell thickness 225 bound between an inner surface 205 and the outer surface 204 (as shown by the two opposing arrows of FIG. 4A), the portion of the cannabinoid molecules 210 are distributed at least partially in the shell thickness 225 and extend inwardly through inner surface 205 and/or extend outwardly through the outer surface 204.

In certain other cannabinoid based capsule embodiments 260, the hollow shell 215 is essentially composed of a biodegradable polymer membrane.

The above sample embodiments loosely directed to FIG. 4C should not be considered limiting to the scope of the invention whatsoever because many more embodiments and variations of embodiments are easily conceived within the teachings, scope and spirit of the instant specification.

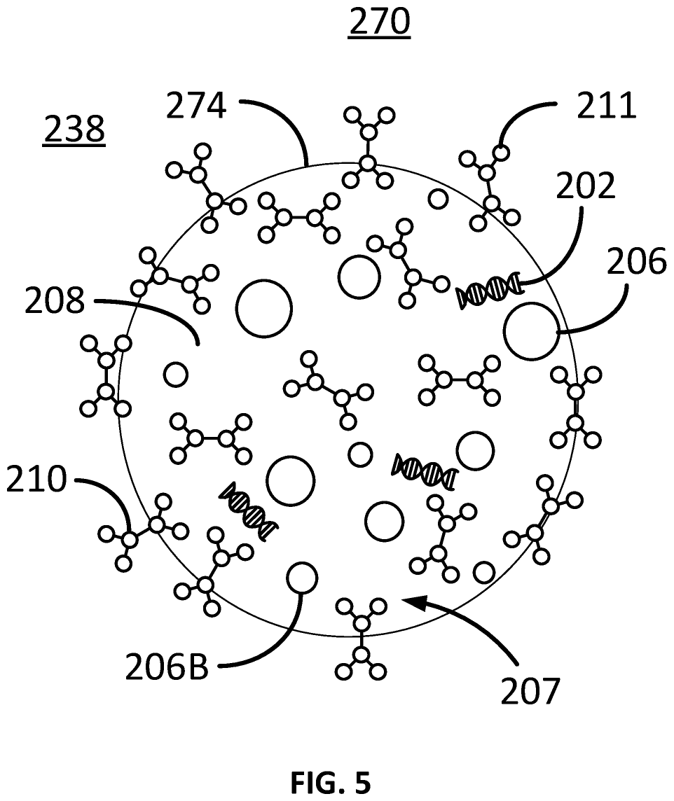

FIG. 5 is a line drawing of a cannabinoid-enriched nano-particle consistent with embodiments of the present invention. Though the discussion of FIG. 5 is directed to a nano-particle embodiment 270, concepts described in conjunction with the nano-particle 270 can equally be applied to a similar micro-particle configuration. The homogenous nano-particle 270 is essentially comprised of target ingredients 208, such as pharmaceutical/medicinal components 206A, markers and/or dyes 206B, genetic material 202, etc., excipient components 207 and at least one kind of cannabinoid molecule 210 dispersed essentially homogeneously throughout the nano-particles 270. In the present embodiment, some of the cannabinoid molecules 210 are exposed at the particle surface 274 thereby exposing the cannabinoid functional end groups 211 to the external environment (outside of the nano-particles 270). Unlike the nano-capsule embodiments 200, 220, 240, 250 and 260, the nano-particle 270 does not have a capsule 215 and core 242 arrangement. Rather, the nano-particle 270 is a homogeneous particle. Certain embodiments envision the cannabinoid molecules 210 extending outwardly from the particle surface 274, as shown. Certain embodiments envision multiple varieties of cannabinoid molecules used in conjunction with a single nano-particle (or nano-capsule for that matter), some of which are depicted in FIGS. 6A-6N. Other embodiments consider fabrication of BBB traversable nano-capsules by mixing lipid soluble solutions with or without cannabinoids together with surfactants containing cannabinoids using ultrasonication or high-pressure homogenization.

Below are some example embodiments illustratively complementing the cannabinoid particle of FIG. 5. The elements called out below are examples provided to assist in the understanding of the present invention and should not be considered limiting.

In that light, one embodiment of the present invention relative to FIG. 5 envisions a cannabinoid based particle 270 comprising a plurality of cannabinoid molecules 210 each possessing a respective functionalized cannabinoid end group 211, at least one active target substance 208, and an excipient 207 that essentially binds the cannabinoid molecules 210 and the active target substance 208 in an aggregate. The excipient 207 essentially defining a particle outer surface 274. A subset of the cannabinoid molecules 210 each positioned with the respective functionalized cannabinoid end group 211 extend from the particle outer surface 274.

The cannabinoid based particle embodiment 270 further imagines the particle 270 being essentially a sphere.

The cannabinoid based particle embodiment 270 further contemplating the cannabinoid molecules 210 comprising at least two different types of cannabinoids.

Optionally, the cannabinoid based particle embodiment 270 further contemplates the particle embodiment 270 is a nanoparticles that has a diameter (or optionally the largest measurement across the capsule) that is less than 1000 nm in diameter, while other embodiments imagine the particles 270 being a micro-particle having a diameter (or largest trans measurement across the capsule) between 1 μm and 2000 μm.

The cannabinoid based particle embodiment 270 further ponders wherein the cannabinoid molecules 210 and the active target substance 208 are essentially evenly dispersed in the particle 270.

The cannabinoid based particle embodiment 270 further considers wherein the active payload 208 is selected from a group comprising at least one of a pharmaceutical drug, a marker, and a genetic nucleic acid molecular sequence 202 (such as DNA, RNA, etc.). This embodiment can be expanded wherein the marker is selected from a group consisting of a radioactive tracer, a dye, a fluorescent dye.

The cannabinoid based particle embodiment 270 further considers wherein the subset of the cannabinoid molecules 210 are partially embedded in the excipient 207.

The cannabinoid based particle embodiment 270 further envisions wherein the excipients 207 is primarily comprised of lipids.

The cannabinoid based particle embodiment 270 further considers wherein the cannabinoid molecules 210 are CBD, as shown in FIG. 6A, or optionally the cannabinoid molecules 210 are made up of at least two different types of cannabinoid molecules, such as CBD and CBC of FIG. 6B.

Certain other embodiments contemplate a functionalized cannabinoid based particle 270 comprising at least one active target substance 208 and cannabinoids 210 interspersed in an excipient binder 207. A particle outer perimeter 274 is defined by an interface where the excipient binder 207 and the active target substance 208 meet an environment outside 238 the excipient binder 207 and the active target substance 208. The cannabinoids 210 possess functionalized cannabinoid end groups 211, which extend outwardly beyond the particle outer perimeter 274.

The functionalized cannabinoid based particle embodiment 270 further imagines the active target substance 208 is selected from a group comprising at least one of a pharmaceutical drug, a marker, and a genetic nucleic acid molecular sequence 202.

The functionalized cannabinoid based particle embodiment 270 further contemplating the cannabinoids 210 comprising at least two different types of cannabinoid molecules 210.

The functionalized cannabinoid based particle embodiment 270 further considers wherein each of the functionalized cannabinoid end groups 211 that extend outwardly beyond the particle outer perimeter 274 are part of a cannabinoid molecule that is at least partially embedded in the excipient binder 207.

Still, yet other embodiments a sub 2000 μm cannabinoid based particle 270 comprising an excipient 207 that essentially binds together a plurality of cannabinoid molecules 210 and at least one active target substance 208 in essentially and evenly dispersed aggregate. Each of the cannabinoid molecules 210 possessing a respective functionalized cannabinoid end group 211 with some of the respective functionalized cannabinoid end groups 211 extending from a particle surface 274 of the particle 270. The particle surface 274 is defined by an outer surface of the excipient 207 where the particle 270 needs an outside environment 238. In the present embodiment, the particle 270 is devoid of a coating (such as a capsule shell 215) at the particle surface 274.

The above sample embodiments relative to FIG. 5 should not be considered limiting to the scope of the invention whatsoever because many more embodiments and variations of embodiments are easily conceived within the teachings, scope and spirit of the instant specification.

Cannabinoids are a diverse class of pharmacologically active chemical compounds that occur naturally in the human body and brain, which are known as endocannabinoids. Exogenous phytocannabinoids are derived from the medicinal plant Cannabis sativa (Cannabaceae family) wherein at least 144 different cannabinoids have been isolated from Cannabis. FIGS. 6A-6N are line drawing diagrams showing the conventional organic chemical structure of 14 common cannabinoids. FIG. 6A depicts cannabidiol molecule (CBD). FIG. 6B depicts a cannabichromene molecule (CBC). FIG. 6C depicts a Δ9-tetrahydrocannabinol molecule (Δ9-THC). FIG. 6D depicts a cannabinol molecule (CBN). FIG. 6E depicts a Δ8-tetrahydrocannabinol molecule (Δ8-THC). FIG. 6F depicts a cannabivarin molecule (CBV). FIG. 6G depicts a Δ9-tetrahydrocannabivarin molecule (THCV). FIG. 6H depicts a cannabigerol molecule (CBG). FIG. 6I depicts a Δ9-Tetrahydrocannabinolic acid molecule (THCV-A). FIG. 6J depicts a cannabidiolic acid molecule (CBDA). FIG. 6K depicts a cannabichromene acid molecule (CBCA). FIG. 6L depicts a Δ9-tetrahydrocannabinol acid molecule (THCA). FIG. 6M depicts a cannabidivarin molecule (CBDV). And, FIG. 6N depicts a cannabigerol acid molecule (CBGA). A generic cannabinoid molecule 210 is depicted in FIG. 3A and is envisioned to assume the structure of a specified cannabinoid molecule, some of which are described in conjunction with FIGS. 6A-6N.

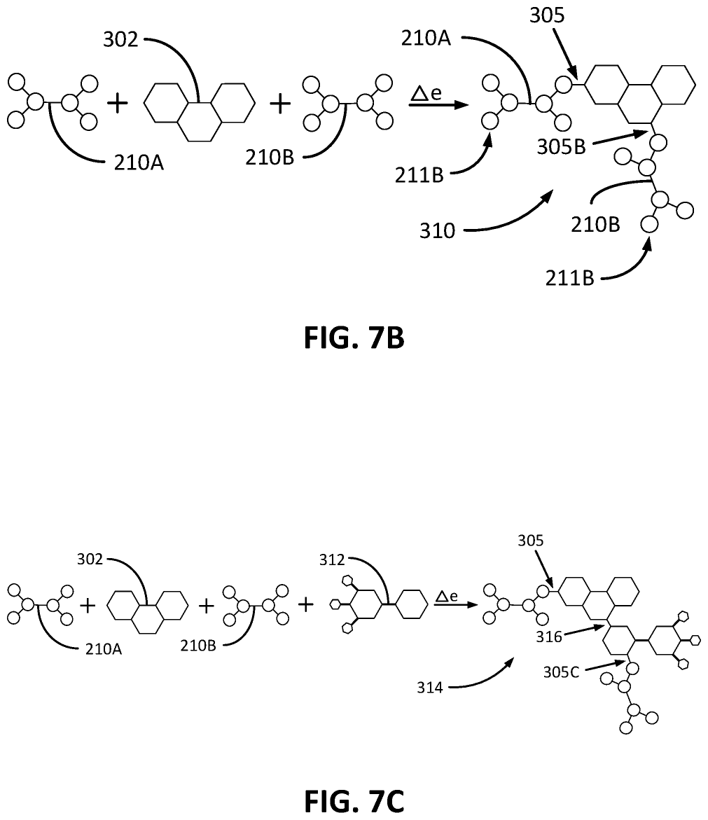

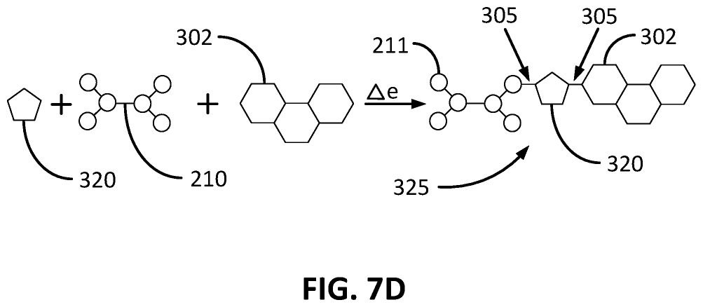

FIGS. 7A-7D are diagrammatic line drawing illustrations of at least one cannabinoid molecule joined or otherwise conjugated with a target molecule consistent with embodiments of the present invention. FIG. 7A is a diagrammatic depiction of a cannabinoid molecule 210 combined with a target molecule 302. More specifically, to the left of the change in energy sign “Δe” is the cannabinoid molecule 210, which can be at least any one of the cannabinoid molecules depicted in FIGS. 6A-6N plus “+” a target molecule 302, which as described above can be a pharmaceutical/medicinal molecule, marker or dye molecule, genetic molecule, etc. The target molecule 302 is depicted as three connected hexagonal blocks to represent a generic target molecule (dye molecule, radiological tracer molecule, pharmacological molecule, genetic coded molecule, etc.). To the right of the change in energy sign (Δe) is the conjugated (joined) 305 single cannabinoid-and-target conjugated molecule 306, or simply “conjugated molecule”. The cannabinoid molecule 210 and the target molecule 302 can be conjugated (connected) by any number of chemical bonds known to those in the chemical arts, such as covalent bonds, ionic bonds, and polar covalent bonds. The conjugated molecule 306 can be introduced to the bloodstream 102 utilizing the functional cannabinoid end group 211 to facilitate transportation across the BBB 115 or other target organs. Crossing the BBB 115 may include utilization of cannabinoid receptors at or on the brain tissue 106 side of the BBB 115. Optionally, the conjugated molecule 306 can be introduced to the lymphatic system to reach the brain or other targeted organs. The conjugated molecule 306 can be carried by an aqueous solution or optionally a lipid-based solution and delivered via a syringe into a vessel, inhalation method (such as intranasal or via the lungs), orally consumed, or optionally introduced at the skin, just to name several examples known to those skilled in the medical arts.

FIG. 7B is a diagrammatic depiction of two cannabinoid molecules 210 conjugated with a target molecule 302 by way of chemical bonds 305 to form a multi-cannabinoid conjugated molecule 310. It should be appreciated by one skilled in the art that the cannabinoid binding sites 305 could be anywhere along the target molecule 302 that would appropriately join with another molecule.

FIG. 7C is a diagrammatic depiction of two cannabinoid molecules 210 conjugated with two different target molecules 302 and 312. The target molecule 312 is diagrammatically depicted as two large hexagonal blocks with three small hexagonal blocks connected to one of the large hexagonal blocks. The conjugated multi-cannabinoid and multi-target molecule 314 is depicted to the right of the change in energy sign (Δe). One skilled in the art will appreciate that many different combinations of conjugated molecules (such as 2 or more similar or dissimilar cannabinoid molecules 210 and/or 2 or more similar or dissimilar target molecules 314) are conceivable within the scope and spirit of the present invention. One advantage of simple conjugated molecules, such as conjugated molecules 306, 310 and 314, is that they may more readily pass through organ barriers, such as the BBB 115.

FIG. 7D is yet another diagrammatic depiction of a cannabinoid molecules 210 conjugated with an active target molecule 320 via an intermediate molecule 320 consistent with embodiments of the present invention. As shown to the left of the change in energy (Δe) arrow an intermediate molecule 320 is added with the cannabinoid molecule 210 and the active target molecule 302. After the molecules 210, 302 and 320 are combined or otherwise bonded together via a change in energy process (Δe), the conjugated molecule 325 is formed. The change in energy process can include a number of chemical reactions known to those skilled in the art including forming the new molecule 325 via a spontaneous reaction or one that requires energy.

Below are some examples illustratively complementing certain embodiments considered in FIGS. 7A-7D. The elements called out below are examples provided to assist in the understanding of the present invention and should not be considered limiting.

In that light, one embodiment of the present invention relative to FIGS. 7A-7D envisions a conjugated cannabinoid based molecule 306 comprising a cannabinoid molecule 210 chemically joined with an active target molecule 302. The conjugated cannabinoid based molecule 306 possessing at least one functionalized cannabinoid end group 211 at a free end 211B of the cannabinoid molecule 210.

The conjugated cannabinoid based molecule 306 embodiment further contemplates the chemical bond is from a set of bonds consisting of a covalent bond, an ionic bond, and a polar covalent bond.

The conjugated cannabinoid based molecule 306 embodiment further contemplating the cannabinoid molecule 210 being chemically joined with the active target molecule 302 via an intermediate molecule 320.

The conjugated cannabinoid based molecule 306 embodiment further pondering the active target molecule 302 being chemically joined 305B with a second cannabinoid molecule 210B wherein the second cannabinoid molecule 210B also comprises at least one functionalized cannabinoid end group 211 at a second free end 211B.

The conjugated cannabinoid based molecule 306 embodiment further considering wherein the active target molecule 302 is chemically joined 316 with a second active target molecule 312. In addition, the second active target molecule 312 and be a different molecule then the active target molecule 302. Optionally, the second active target molecule 312 and the chemically joined 305C with a second cannabinoid molecule 210B.

The conjugated cannabinoid based molecule 306 embodiment further imagining the active target molecule 302 being selected from a group comprising at least one of a pharmaceutical drug molecule, a marker molecule, and a genetic nucleic acid molecular sequence molecule 202. In certain examples, the marker is selected from a group consisting of a radioactive tracer, a dye, and a fluorescent dye.

The above sample embodiments should not be considered limiting to the scope of the invention whatsoever because many more embodiments and variations of embodiments are easily conceived within the teachings, scope and spirit of the instant specification.

It is to be understood that even though numerous characteristics and advantages of various embodiments of the present invention have been set forth in the foregoing description, together with the details of the structure and function of various embodiments of the invention, this disclosure is illustrative only, and changes may be made in detail, especially in matters of structure and arrangement of parts within the principles of the present invention to the full extent indicated by the broad general meaning of the terms in which the appended claims are expressed. For example, though certain capsule and/or particle configurations are shown independently, someone in possession of the concepts described herein will readily understand combining specific aspects described in conjunction with one figure as applied to concepts described in conjunction with another figure. For example, benefits of a cannabinoid functionalized end group facilitating crossing organs, such as the BBB 115, can be applied throughout the different embodiments of the present invention. Additionally, conceptually applied techniques to manufacture the shell of a nano-capsule can be more or less equally applied to all of the capsule embodiments disclosed herein, notwithstanding the subtle variations specifically described in the different embodiments. Also, it should be further appreciated that though nano-particles and nano-capsules are described herein, these concepts can be equally applied to micro-capsules or micro-particles or optionally larger structures without departing from the scope and spirit of the present invention. In addition, those surfactants and lipids are described as structural components of certain capsule or particle embodiments, it should be appreciated that other in vivo biodegradable substances can be equally used within the concepts described in the present invention. Further, the terms “one” is synonymous with “a”, which may be a first of a plurality.

It will be clear that the present invention is well adapted to attain the ends and advantages mentioned as well as those inherent therein. While presently preferred embodiments have been described for purposes of this disclosure, numerous changes may be made which readily suggest themselves to those skilled in the art and which are encompassed in the spirit of the invention disclosed.

What is claimed is:

1. A capsule comprising:
an in vivo dissolvable hollow shell defined by a shell thickness bound between an outer surface and an inner surface;
a plurality of cannabinoid molecules dispersed in the hollow shell, at least one of the cannabinoid molecules is at least partially disposed in the shell thickness and at least partially exposed at the outer surface; and
an inner core containing excipients and an active payload, the inner core encapsulated in the hollow shell.

2. The capsule of claim 1 wherein the at least one cannabinoid molecule comprising a functionalized cannabinoid end group that is at least partially exposed at the outer surface.

3. The capsule of claim 1 further comprising at least a second of the cannabinoid molecules comprising a functionalized cannabinoid end group extending from the outer surface.

4. The capsule of claim 1 further comprising at least a second of the cannabinoid molecules extending inwardly from the inner surface.

5. The capsule of claim 1 wherein the hollow shell is essentially composed of a surfactant mixed with the cannabinoid molecules.

6. The capsule of claim 1 wherein the active payload is selected from a group comprising at least one of a pharmaceutical drug, a marker, and a genetic nucleic acid molecular sequence.

7. The capsule of claim 6 wherein the marker is selected from a group consisting of a radioactive tracer, a dye, and a fluorescent dye.

8. The capsule of claim 1 wherein the capsule is a nano-capsule that is less than 1000 nm in diameter.

9. The capsule of claim 1 wherein the capsule is a micro-capsule that is between 1 μm and 2000 μm.

10. The capsule of claim 1 wherein the plurality of cannabinoid molecules are geometrically organized within the hollow shell wherein at least some of the cannabinoid molecules extend from the outer surface exposing functionalized end-groups of the cannabinoid molecules.

11. The capsule of claim 1 wherein the cannabinoid molecules comprise CBD.

12. The capsule of claim 1 wherein none of the cannabinoid molecules are entirely in the inner core.

13. A method for manufacturing a cannabinoid capsule, the method comprising:
providing a base mixture of cannabinoid molecules, capsule forming molecules, excipients, and at least one active target substance;
energizing the base mixture until the capsule forming molecules generate a shell encapsulating a core, the core comprising the cannabinoid molecules, the excipients, and the at least one active target substance, and
the cannabinoid molecules are distributed throughout the core, embedded in the shell, and at least partially extend outside of a shell outer surface of the shell.

14. The method of claim 13 further comprising performing the energizing step until the shell is less than 1000 nm in diameter.

15. The method of claim 13 wherein the cannabinoid molecules each comprise a functionalized cannabinoid end group, at least some of the functionalized cannabinoid end groups extend outside of the shell outer surface.

16. The method of claim 13 wherein the cannabinoid molecules comprise at least two different cannabinoids.

17. The method of claim 13 wherein the energizing step includes sonicating the base mixture, heating and cooling the base mixture over multiple cycles, agitating the base mixture, and subjecting the base mixture to acid.

18. A cannabinoid based particle comprising:
a plurality of cannabinoid molecules each possessing a respective functionalized cannabinoid end group;
at least one active target substance;
an excipient that essentially binds together the cannabinoid molecules and the active target substance in an aggregate, the excipient essentially defining a particle outer surface; and
a subset of the cannabinoid molecules each positioned with the respective functionalized cannabinoid end group extending from the particle outer surface.

19. The cannabinoid based particle of claim 18 wherein the cannabinoid molecules and the active target substance are essentially evenly dispersed in the particle.

20. The cannabinoid based particle of claim 18 wherein the subset of the cannabinoid molecules are partially embedded in the excipient.
